(19) United States
(12) Patent Application Publication
(10) Pub. No.: US 2026/0248544 A1
(43) Pub. Date: Aug. 27, 2026

(54) MULTI-CONFIGURABLE STERNAL FIXATION SYSTEMS, METHODS, AND DEVICES WITH A FORCE DISPERSION SUPPLEMENT

(71) Applicant: ThoraGenix Innovations, Inc., Tampa, FL (US)

(72) Inventors: Frederick J. Thabet, Boca Raton, FL (US); Ravi Kanagala, Boca Raton, FL (US); Keri George, Calgary (CA)

(73) Assignee: ThoraGenix Innovations, Inc., Tampa, FL (US)

(21) Appl. No.: 19/345,318

(22) Filed: Sep. 30, 2025

Related U.S. Application Data
(60) Provisional application No. 63/701,325, filed on Sep. 30, 2024.

(51) Int. Cl. A61B 17/82; A61B 17/56; A61B 17/68

(52) U.S. Cl. CPC A61B 17/823; A61B 2017/564; A61B 2017/681

(57) ABSTRACT

Systems, methods, and devices disclosed herein include a sternal fixation device involving a sternal fixation wire with one or more force dispersion supplement(s). The sternal fixation wire is operable to at least partially wrap around a first bone section and a second bone section to provide a compressive force on the first bone section and the second bone section. The compressive force pushes the first bone section and the second bone section toward a centerline between the first bone section and the second bone section. Additionally, the force dispersion supplement is disposed around at least a portion of the sternal fixation wire. The force dispersion supplement is operable to increase a force dispersion surface area of the sternal fixation wire at attachment zone(s) on the first bone section or the second bone section. The increased force dispersion surface area reduces potential damage caused to the bone tissue.

### CROSS REFERENCE TO RELATED APPLICATION

This application claims priority to U.S. Provisional Patent Application Ser. No. 63/701,325, filed Sep. 30, 2024, and titled “MULTI-CONFIGURABLE STERNAL FIXATION SYSTEMS, METHODS, AND DEVICES WITH A FORCE DISPERSION SUPPLEMENT,” which is incorporated by reference herein in its entirety.

### BACKGROUND

In a sternotomy procedure, a surgeon severs a patient's sternum into two separate portions to access thoracic organs such as the heart, lungs, and/or blood vessels. After accessing the thoracic organs, the two portions of the sternum are rejoined. The ideal goal is complete rejoining of the sternal portions with new bone growth in the absence of complications. Unfortunately, patient recovery from a conventional sternotomy is often slow and problematic. As the two sections of the sternum are brought back together by the surgeon, proper compression and a tight realignment of the end plates of the sternal surfaces is rarely achieved resulting in non-union or malunion of the separated sternal halves. Non-union allows for motion such as sliding of one sternal half against the other sternal half, leading to significant pain for the patient and increased chance of infection. Additionally, the lack of proper compression leads to the formation of fibrous scar tissue instead of the desired new bone. If further surgical procedures are required, the scar tissue must be removed further complicating the procedure.

It is with these observations in mind, among others, that various aspects of the present disclosure were conceived and developed.

### SUMMARY

Systems, methods, and devices disclosed herein can address the aforementioned problems. For instance, a sternal fixation system can include a sternal fixation wire operable to at least partially wrap around a first bone section and a second bone section. The sternal fixation wire can provide a compressive force on the first bone section and the second bone section which squeezes, biases, compresses, and/or pushes the first bone section and the second bone section toward a centerline between the first bone section and the second bone section. Additionally, the sternal fixation system can include a force dispersion supplement disposed around at least a portion of the sternal fixation wire. The force dispersion supplement can be operable to increase a force dispersion surface area of the sternal fixation wire at one or more attachment zone(s) on the first bone section and/or the second bone section. In this way, the sternal fixation system can optimize a bone healing procedure following a sternotomy by reducing damage to the first bone portion and/or a second bone portion caused by the pressure of the sternal fixation wire.

In some examples, the force dispersion supplement can include at least a flexible sleeve wrapped around the at least the portion of the sternal fixation wire. Additionally or alternatively, the force dispersion supplement can include at a least a mesh pad. The mesh pad can include an interior space which permits bodily fluids, medication, and/or blood to flow through the mesh pad. The force dispersion surface area can be increased, by the force dispersion supplement, at a sternal edge of a sternum body between a first costal notch and a second costal notch. Moreover, the force dispersion surface area can be increased, by the force dispersion supplement, at a sternal edge of a manubrium between an articular facet for rib 1 and an articular demifacet for rib 2.

In some instances, the force dispersion supplement can cover or conceal a full circumferential surface of the at least the portion of the sternal fixation wire. Also, the force dispersion supplement can cover or conceal a first side of a circumferential surface of the at least the portion of the sternal fixation wire, the first side mating with and/or being adjacent to an outer surface of the first bone section or the second bone section; and/or the forces dispersion supplement can expose a second side of the circumferential surface opposite the first side.

In some scenarios, a sternal fixation system can include one or more sternal fixation wires operable to at least partially wrap around a first bone section of a sternum and a second bone section of the sternum to provide a compressive force to the first bone section and the second bone section. The compressive force can be applied to one or more attachment zones between one or more pairs of costal notches of the sternum. Furthermore, the sternal fixation system can include one or more force dispersion supplements disposed around one or more portion of the one or more sternal fixation wire. The one or more force dispersion supplements can increase a force dispersion surface area applied to the one or more attachment zones. Additionally, the one or more sternal fixation wires can include a first sternal fixation wire having a first length corresponding to a first attachment zone of the one or more attachment zones. The first attachment zone can be at least partially between a first costal notch and a second costal notch. Also the one or more sternal fixation wires can include a second sternal fixation wire having a second length corresponding to a second attachment zone of the one or more attachment zones. The second attachment zone can be at least partially between the second costal notch and a third costal notch. Moreover, the one or more sternal fixation wires can include a third sternal fixation wire having a third length corresponding to a third attachment zone of the one or more attachment zones. The third attachment zone can be at least partially between the third costal notch and a fourth costal notch.

Furthermore, in some examples, the first length of the first sternal fixation wire can be greater than the second length of the second sternal fixation wire. Also, the one or more force dispersion supplements can include a first force dispersion supplement disposed on the first sternal fixation wire, and/or a second force dispersion supplement disposed on the second sternal fixation wire. Additionally, the third length of the third sternal fixation wire can be greater than the second length of the second sternal fixation wire, and/or the one or more force dispersion supplements can include a third force dispersion supplement disposed on the third sternal fixation wire.

In some instances, the one or more sternal fixation wires can include a plurality of sternal fixation wires; and/or individual sternal fixation wires of the plurality of sternal fixation wires can include a first force dispersion supplement of the one or more force dispersion supplements corresponding to a first sternal edge of the first bone section. The individual sternal fixation wires of the plurality of sternal fixation wires can also include a second force dispersion supplement of the one or more force dispersion supplements corresponding to a second sternal edge of the second bone section. Furthermore, the one or more sternal fixation wires can include one sternal fixation wire with two force dispersion supplements spaced a selectively determined distance apart along the one sternal fixation wire. The one sternal fixation wire can be a first sternal fixation wire, the distance can be a first distance, and the sternal fixation system can include a second sternal fixation wire with two force dispersion supplements spaced a second distance apart along the second sternal fixation wire. The second distance can be different than the first distance.

In some scenarios, a sternal fixation method includes wrapping one or more sternal fixation wires around a first bone section of a sternum and a second bone section of the sternum to apply a compressive force to one or more target areas between one or more pairs of costal notches of the sternum. The sternal fixation method can also include distributing the compressive force by using one or more force dispersion supplements, disposed at least partially around the one or more sternal fixation wires, to create an increased force dispersion surface area. Moreover, the sternal fixation method can include preventing damage to the first bone section or the second bone based on the increased force dispersion surface area. For instance, the increased force dispersion surface area can reduce the amount of force on any particular contact point caused by the compressive force.

In some examples, the wrapping of the one or more sternal fixation wires can include orienting the one or more sternal fixation wires to mate the one or more force dispersion supplements with one or more attachment zones of the first bone section or the second bone section. Also, the method can include selectively covering or concealing a first portion of the one or more sternal fixation wires with the one or more force dispersion supplements, and/or selectively exposing a second portion of the one or more sternal fixation wires. Additionally, the method can include tying the one or more sternal fixation wires such that the first bone section is immobilized relative to the second bone section. Moreover, the wrapping of the one or more sternal fixation wires around the first bone section and the second bone section can include positioning a first force dispersion supplement of the one or more force dispersion supplements at a first attachment zone the first bone section including a first sternal edge, and/or positioning a second force dispersion supplement of the one or more force dispersion supplements at a second attachment zone of the second bone section including a second sternal edge, across a center midline of the sternum from the first attachment zone.

### BRIEF DESCRIPTION OF THE DRAWINGS

FIG. 1 depicts an example system including a sternal fixation wire with a force dispersion supplement for healing two bone portions together.

FIG. 2A depicts an example system including a sternal fixation wire with a force dispersion supplement covering or concealing a full circumferential surface of a covered portion of the wire.

FIG. 2B depicts an example system including a sternal fixation wire with a force dispersion supplement covering a partial circumferential surface of a covered portion of the wire.

FIG. 3A depicts an example system including a sternal fixation wire with two force dispersion supplements spaced a predetermined distance apart and with full circumferential surface coverage.

FIG. 3B depicts an example system including a sternal fixation wire with two force dispersion supplements spaced a predetermined distance apart and with partial circumferential surface coverage.

FIG. 3C depicts an example system including a sternal fixation wire with a plurality of force dispersion supplements spaced a plurality of predetermined distances apart and with full circumferential surface coverage.

FIG. 3D depicts an example system including a sternal fixation wire with a plurality of force dispersion supplements spaced a plurality of predetermined distances apart and with partial circumferential surface coverage.

FIG. 4 depicts an example method of sternal fixation which can be performed by any of the systems depicted in FIGS. 1-3D.

### DETAILED DESCRIPTION

It will be appreciated that numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

The systems, methods, and devices disclosed herein can include a sternal fixation device which improves a healing procedure for rejoining two portions of severed bone. The sternal fixation device can include a sternal fixation wire designed to wrap around the two portions of bone, and one or more force dispersion supplements operable to reduce damage at the points of contact on the bone tissue. For instance, the sternal fixation wire can at least partially wrap around a first bone section of a sternum and a second bone section of the sternum to provide a compressive force on the first bone section and the second bone section. The compressive force can push the first bone section and the second bone section toward a centerline between the first bone section and the second bone section. Additionally, the force dispersion supplement(s) can be disposed around one or more portions of the sternal fixation wire corresponding to attachment zones on the bone sections. The force dispersion supplement(s) can increase a force dispersion surface area of the sternal fixation wire at the attachment zones on the first bone section and/or the second bone section.

In scenarios where a high compressive force is needed to effectively rejoin and heal the two bone, the increased force dispersion surface area can reduce damage caused to the bone tissue which may otherwise occur due to the hardness of the sternal fixation wire. The force dispersion supplements can prevent the sternal fixation wire from cutting into the bone tissue following a sternotomy. This improved distribution of stress on the bone can prevent shearing, and bone erosion, while also preventing wire migration. Moreover, the sternal fixation device disclosed herein can include various configurations to better distribute the mechanical stress by maximizing surface area coverage of the lateral compressive. A surgeon can selectively configure the sternal fixation device to meet the needs of any particular patient anatomy. This technology can be useful in situations involving low density bones (e.g., caused by osteoporosis) which can be more susceptible to damage from the sternal fixation wires. The disclosed systems can result in improved bone securement strength, reduced bone tissue shearing, and/or reduced wire movement.

Moreover, the sternal fixation device can be used to retrofit a previous sternal fixation device, for instance, by adding force dispersion supplements to a previously implanted wire, and/or by removing the previously implanted wire and replacing it with one including sternal fixation supplement(s). Furthermore, in some scenarios, medication, adhesives, and/or a bone growth filler including bone growth stimulation agents can be added to the force dispersion supplement to further improve patient outcomes.

Additional benefits and advantages of the disclosed technology will become apparent from the detailed description below.

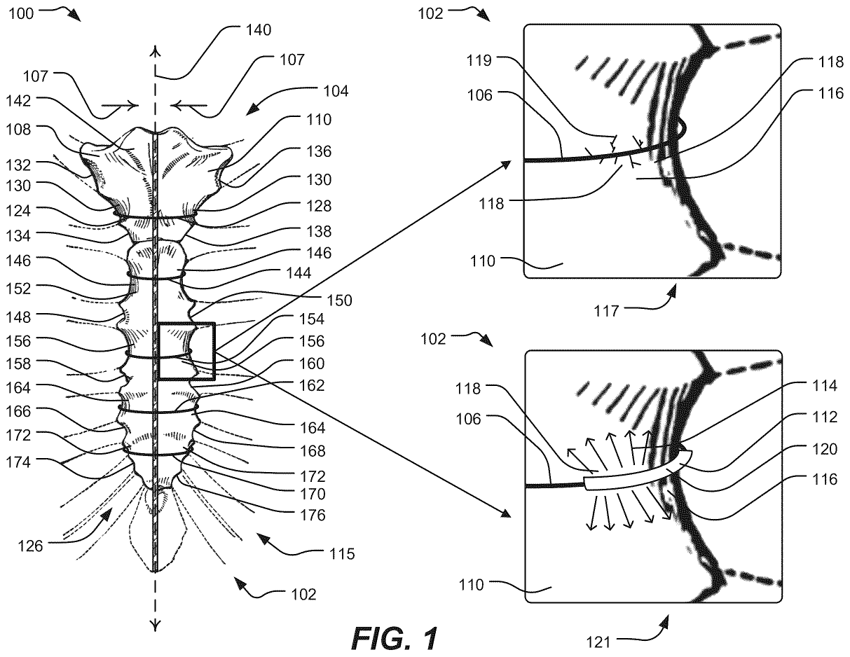

FIG. 1 illustrate an example bone fixation system 100 including a sternal fixation device 102 for bringing and securing together two sections of a sternum 104 following a sternotomy. The sternal fixation device 102 can include one or more sternal fixation wire(s) 106 which are operable to wrap around a first bone section 108 and a second bone section 110. Furthermore, the sternal fixation device 102 can include one or more force dispersion supplement(s) 112. The force dispersion supplement(s) 112 can include components disposed on the sternal fixation wire(s) 106 which increase a force dispersion surface area 114 of the sternal fixation wire(s) 106 to optimize a placement and/or securement procedure 115 and/or a healing process, as discussed herein.

In some examples, the sternal fixation device 102 can include the sternal fixation wire 106 which omits the force dispersion supplement 112, as shown in one scenario 117. In this scenario 117, since the sternal fixation wire 106 is harder and denser than the bone tissue 116 at the attachment zones 118 of the first bone section 108 and/or second bone section 110, a surgeon may attempt to avoid overtightening the sternal fixation wire 106 because doing so could cause a stress riser against the bone. Furthermore, excessive tightening can cause the sternal fixation wire 106 to tear through the bone tissue causing damage 119 to the first bone section 108 and/or the second bone section 110, either during the device placement and/or securement procedure 115, and/or post-operatively during a healing process as the patient moves about. Because of this, the amount of horizontal compression force 107 applied to the cut bone portions 108 and 110 may not be sufficient for full union and healing of the two bone sections 108 and 110 (e.g., in accordance with Wolff's Law).

In another scenario 121, the sternal fixation device 102 can include the force dispersion supplement(s) 112. The force dispersion supplement(s) 112 can include material formed onto at least a portion 120 of the sternal fixation wire 106. This can include a different material than the hard metal of the sternal fixation wire 106, such as a polymer material, silicon, a fibrous mesh material, and/or a softer type of metal material. In some examples, the force dispersion supplement 112 can include a coating, such as a silicon coating. Alternatively, the force dispersion supplement(s) 112 can be formed of a same material as the sternal fixation wire 106.

In some instances, the force dispersion supplement(s) 112 can increase the force dispersion surface area 114 of the sternal fixation device 102, for instance, by increasing a diameter dimension and/or a width dimension of the sternal fixation wire 106. As the compressive force 107 is applied with the sternal fixation wire 106, the force dispersion supplement(s) 112 can be pressed against the first bone section 108 and/or the second bone section 110 at one or more attachment zones 118. This can cause the force dispersion supplement(s) 112 to flatten and/or expand (e.g., in scenarios involving flexible/elastic force dispersion supplement(s) 112), which can further increases the force dispersion surface area 114 at the attachment zone(s) 118. As such, the sternal fixation device 102 can have increased surface contact with the attachment zones 118, which decreases the amount of force experienced at any individual contact point of the attachment zones 118. Accordingly, the surgeon can eliminate gapping and loosening of the sternal fixation wires 106 by providing an adequate compressive force 107 with the sternal fixation wire 106 to secure the first bone section 108 and the second bone section 110 together for proper healing while decreasing an amount of damage 119 caused to the bone tissue 116 by the sternal fixation wire(s) 106.

In some examples, the sternal fixation device 102 can include a plurality of sternal fixation wires 124. Individual sternal fixation wires 106 of the plurality of sternal fixation wires 124 can correspond to different anatomical locations on the target bone 126, for instance, by having particular length dimensions, width dimensions, material hardnesses, or so forth, which matches the dimensions, contours, and/or bone density at the particular target locations. The plurality of sternal fixation wires 124 can include a first sternal fixation wire 128 corresponding to and/or operable for placement at one or more first attachment zone(s) 130. The first attachment zone(s) 130 can be between a first costal notch 132 for rib 1 and a second costal notch 134 for rib 2 on the first bone section 108. Additionally, the first attachment zone(s) 130 can be between a third costal notch 136 for rib 1 and a fourth costal notch 138 for rib 2 on the second bone section 110. These first attachment zones 130 can be located across a center midline 140 from each other, the center midline 140 dividing the first bone section 108 and the second bone section 110. The first attachment zone(s) 130 can include lateral borders and/or outer sternal edges at the manubrium 142 (e.g. between an articular facet for rib 1 and an articular demifacet for rib 2). The compressive force 107 applied by the plurality of sternal fixation wires 106 can push, pull, squeeze, bias, and/or compress the first bone section 108 and/or the second bone section 110 toward the center midline 140 by distributing the compressive force 107 at least at the first attachment zone(s) 130.

Furthermore, in some instances, the plurality of sternal fixation wires 124 can include a second sternal fixation wire 144 corresponding to and/or operable for placement at one or more second attachment zone(s) 146. The second attachment zone(s) 146 can be a second attachment zone 146 between the second costal notch 134 for rib 2 and a fifth costal notch 148 for rib 3 on the first bone section 108. Additionally, the second attachment zone(s) 146 can be between the fourth costal notch 138 for rib 2 and a sixth costal notch 150 for rib 3 on the second bone section 110. These second attachment zone(s) 146 can be located across the center midline 140 from each other, and can include the lateral borders and/or outer edges of the sternum body 152. The compressive force 107 applied by the plurality of sternal fixation wires 106 can push, pull, squeeze, bias, and/or compress the first bone section 108 and/or the second bone section 110 toward the center midline 140 by distributing the compressive force 107 at least at the second attachment zones 146.

Also, the plurality of sternal fixation wires 124 can include a third sternal fixation wire 154 corresponding to and/or operable for placement at one or more third attachment zone(s) 156. The third attachment zone(s) 156 can include a third attachment zone 156 between the fifth costal notch 148 for rib 3 and a seventh costal notch 158 for rib 4 on the first bone section 108. Additionally, the third attachment zone(s) 156 can be between the sixth costal notch 150 for rib 3 and an eight costal notch 160 for rib 4 on the second bone section 110. These third attachment zone(s) 156 can be located across the center midline 140 from each other, and can include the lateral borders and/or outer edges of the sternum body 152. The compressive force 107 applied by the plurality of sternal fixation wires 106 can push, pull, squeeze, bias, and/or compress the first bone section 108 and/or the second bone section 110 toward the center midline 140 by distributing the compressive force 107 at least at the third attachment zones 156.

The plurality of sternal fixation wires 124 can also include a fourth sternal fixation wire 162 corresponding to and/or operable for placement at one or more fourth attachment zone(s) 164. The fourth attachment zone(s) 164 can include a fourth attachment zone 164 between the seventh costal notch 158 for rib 4 and a ninth costal notch 166 for rib 5 on the first bone section 108. Additionally, the fourth attachment zone(s) 164 can be between the eight costal notch 160 for rib 4 and a tenth costal notch 168 for rib 5 on the second bone section 110. These fourth attachment zone(s) 164 can be located across the center midline 140 from each other, and can include the lateral borders and/or outer edges of the sternum body 152. The compressive force 107 applied by the plurality of sternal fixation wires 106 can push, pull, squeeze, bias, and/or compress the first bone section 108 and/or the second bone section 110 toward the center midline 140 by distributing the compressive force 107 at least at the fourth attachment zones 164.

Additionally, the plurality of sternal fixation wires 124 can also include a fifth sternal fixation wire 170 corresponding to and/or operable for placement at one or more fifth attachment zone(s) 172. The fifth attachment zone(s) 172 can include a fifth attachment zone 172 between the ninth costal notch 166 for rib 5 and an eleventh costal notch 174 for rib 6 on the first bone section 108. Additionally, the fifth attachment zone(s) 176 can be between the tenth costal notch 168 for rib 5 and a twelfth costal notch 176 for rib 6 on the second bone section 110. These fifth attachment zone(s) 172 can be located across the center midline 140 from each other, and can include the lateral borders and/or outer edges of the sternum body 152. The compressive force 107 applied by the plurality of sternal fixation wires 106 can push, pull, squeeze, bias, and/or compress the first bone section 108 and/or the second bone section 110 toward the center midline 140 by distributing the compressive force 107 at least at the fifth attachment zones 172.

Although the first sternal fixation wire 128, the second sternal fixation wire 144, the third sternal fixation wire 154, the fourth sternal fixation wire 162, and the fifth sternal fixation wire 170 are discussed above as separate wires, it is to be understood that these sternal fixation wires 106 can be combined, for instance, into a single wire, two wires, or so forth. In these scenarios one sternal fixation wire 106 can be used to attach to multiple attachment zones (e.g. the first attachment zone(s) 130, the second attachment zone(s) 146, the third attachment zone(s) 156, the fourth attachment zone(s) 164, the fifth attachment zone(s) 172, and/or any combination thereof), for instance, using a spiraling or crisscrossing configuration along a length of the target bone 126.

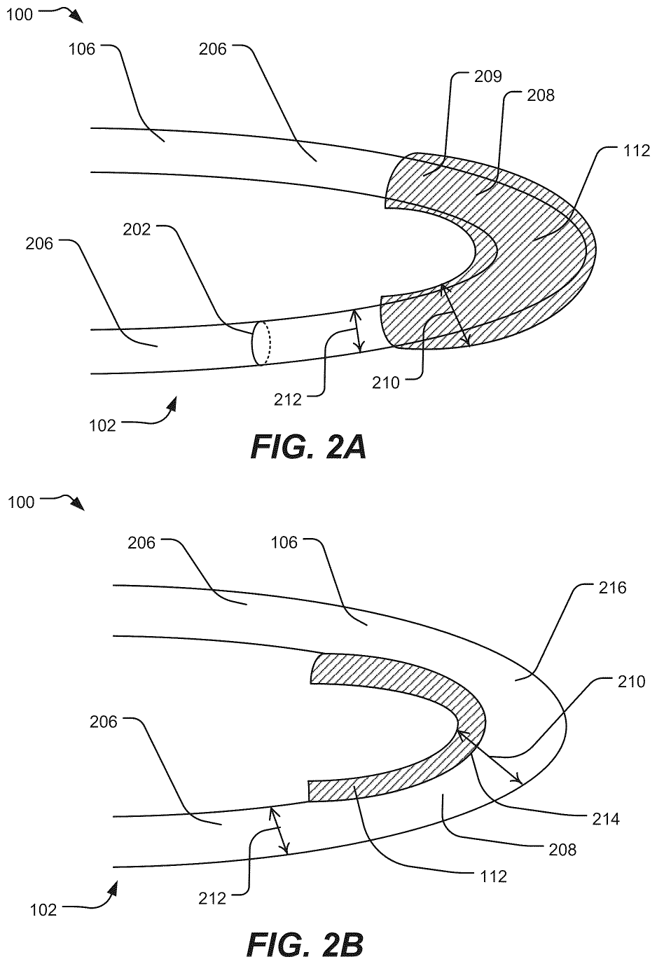

FIGS. 2A and 2B depict example bone fixation system(s) 100 including the sternal fixation device 102 with the sternal fixation wire(s) 106 and the force dispersion supplement(s) 112. The bone fixation system(s) 100 depicted in FIGS. 2A and 2B can be similar to, identical to, and/or can form at least a portion of the bone fixation system(s) 100 depicted in FIG. 1.

As shown in FIG. 2A, the force dispersion supplement(s) 112 can cover or conceal a full circumferential surface 202 of at least a portion 204 of the sternal fixation wire 106. The sternal fixation wire 106 can include one or more bare portion(s) 206 which omit the force dispersion supplement(s) 112 and/or one or more supplemented portion(s) 208 which include the force dispersion supplement(s) 112 covering or concealing the full circumferential surface 202 of the supplemented portion 208. The force dispersion supplement 112 can be a sleeve 209 permanently affixed to the sternal fixation wire 106 or slidable along the sternal fixation wire 106. The supplemented portion(s) 208 of the sternal fixation wire 106 can have the force dispersion supplement 112 wrapped fully around the circumference of the sternal fixation wire 106. Accordingly, the supplemented portion(s) 208 can have a first diameter dimension 210 which is expanded and/or greater than a second diameter dimension 212 of the bare portion(s) 206, which increases the force dispersion area 114. Furthermore, the supplemented portion(s) 208 can be selectively arranged along a length of the sternal fixation wire 106 such that the supplemented portion(s) 208 align with an outer sternal edge and/or other high-pressure contact points of the first bone section 108 and/or the second bone section 110. In some instances, the force dispersion supplement 112 can cover or conceal an entire length of the sternal fixation wire 106 so that the sternal fixation wire 106 does not include bare portion(s) 206. It is to be understood that the various configurations disclosed herein can be selected by the surgeon based on the particular anatomical structures of the bones being healed together. For instance, the sternal fixation system 102 can include a plurality of sternal fixation wires 106 having these different configurations, for instance in a group or pack. Moreover, other types of bone sections beyond the sternum can be adjoined using the technology disclosed herein.

As shown in FIG. 2B, the force dispersion supplement(s) 112 can only cover or conceal a side of the sternal fixation wire 106 at the supplemented portion(s) 208. For instance, a concealed or covered side 214 of the supplemented portion 208 can include the force dispersion supplement 112 and a bare side 216 of the supplemented portion 208 can omit the force dispersion supplement 112. In this configuration, the force dispersion supplement 112 can cover or conceal less than 100% of the circumferential surface 210, such that the circumference of the supplemented portion 208 is only partially concealed or partially covered by the force dispersion supplement 112. The covered side 214 can be directed towards the attachment zone(s) 118 (e.g., during an orientation operation performed by the surgeon), while the bare side 216 can be directed away from the attachment zone(s) 118. The covered side 214 can be an interior side 218 while the bare side 216 can be an exterior side 220 of the sternal fixation wire 106. Also, the covered side 214 can be an opposite side from the bare side 216, for instance, located across the first diameter 210 from the bare side 216. In some instances, the force dispersion supplement 112 can include a partial or half sleeve, or a pad, such as a mesh, interwoven, and/or fibrous pad.

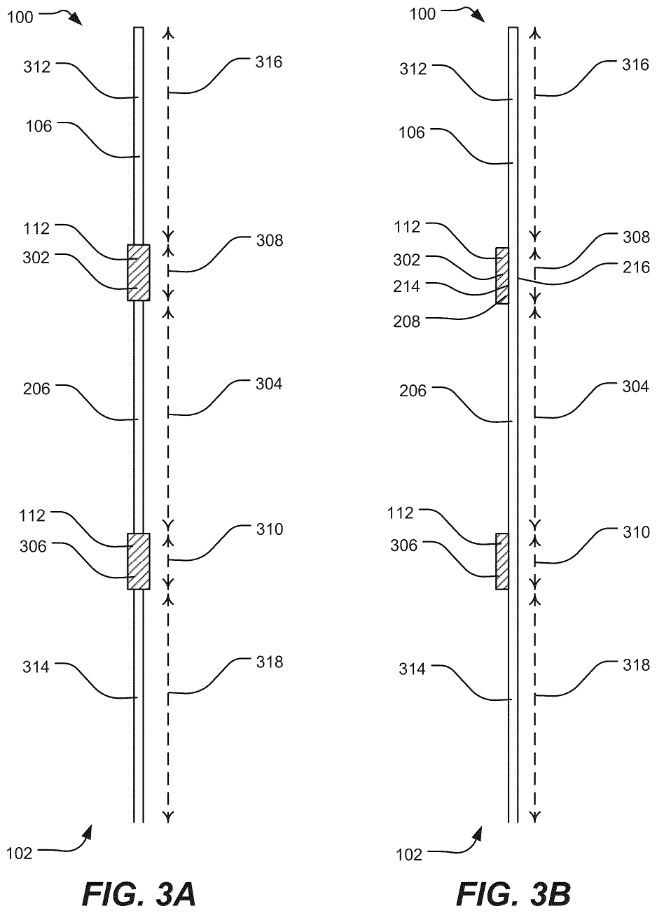

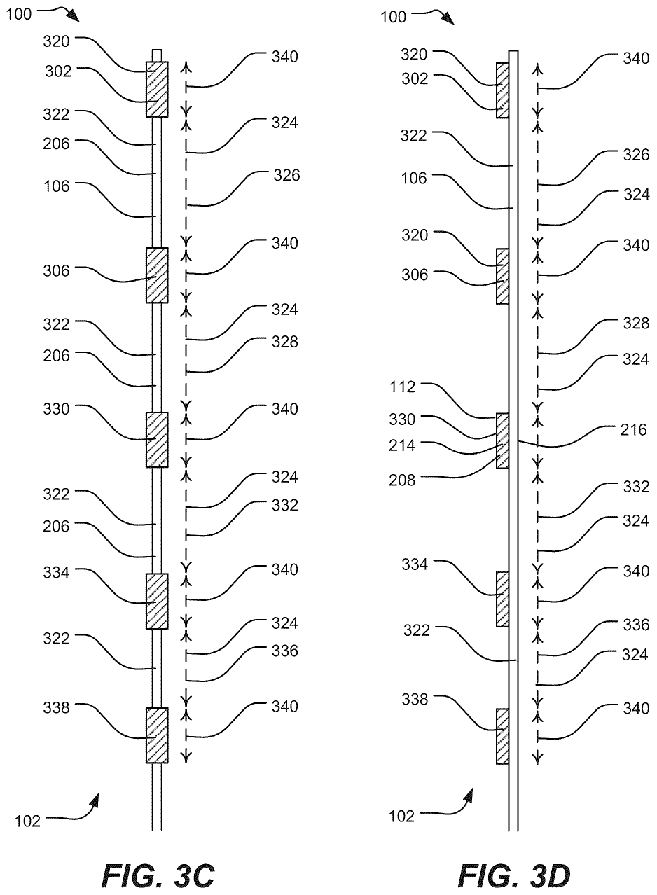

FIGS. 3A-3D depict example bone fixation system(s) 100 including a sternal fixation wire 106 with a plurality of force dispersion supplements 112. The bone fixation system(s) 100 depicted in FIGS. 3A-3D can be similar to, identical to, and/or can form at least a portion of the bone fixation system(s) 100 depicted in FIG. 1.

Turning to FIGS. 3A and 3B, in some instances, the bone fixation system 100 can include two force dispersion supplements 112, such as a first force dispersion supplement 302 spaced a predetermined distance 304 apart from a second force dispersion supplement 306. The first force dispersion supplement 302 can have a first length dimension 308 and/or the second force dispersion supplement 306 can have a second length dimension 310 corresponding to one or more dimension(s) of the attachment zone(s) 118 for which the sternal fixation wire 106 is configured. In some instances, the sternal fixation wire 106 can be the first sternal fixation wire 128 corresponding to the first attachment zone(s) 130 on the manubrium 142. In this case, the predetermined distance 306 between the first force dispersion supplement 302 and the second force dispersion supplement 306 (e.g., the bare portion 206) can be a length which matches a lateral dimension of the manubrium 142 between the first attachment zones 130. Also, the first length dimension 308 of the first force dispersion supplement 302 and/or the second length dimension 310 of the second force dispersion supplement 306 can match a dimension/contours of the first attachment zones 130 of the outer sternal edges of the manubrium 142.

In some examples, the sternal fixation wire 106 can include the second sternal fixation wire 144, such that the predetermined distance 304 corresponds to a lateral dimension across the sternum body 152 between the second attachment zones 146, and/or the first length dimension 308 or the second length dimension 310 can match dimensions/contours of the outer sternal edges at the second attachment zones 146. Moreover, the sternal fixation wire 106 can include the third sternal fixation wire 154, such that the predetermined distance 304 corresponds to a lateral dimension across the sternum body 152 between the third attachment zones 154, and/or the first length dimension 308 or the second length dimension 310 can match dimensions/contours of the outer sternal edges at the third attachment zones 154. The sternal fixation wire 106 can also include the fourth sternal fixation wire 162, such that the predetermined distance 304 corresponds to a lateral dimension across the sternum body 152 between the fourth attachment zones 164, and/or the first length dimension 308 or the second length dimension 310 can match dimensions/contours of the outer sternal edges at the fourth attachment zones 164. Furthermore, the sternal fixation wire 106 can include the fifth sternal fixation wire 170, such that the predetermined distance 304 corresponds to a lateral dimension across the sternum body 152 between the fifth attachment zones 172, and/or the first length dimension 308 or the second length dimension 310 can match dimensions/contours of the outer sternal edges at the fifth attachment zones 172. As shown in FIG. 3B, the first dispersion supplement 302 and/or the second dispersion supplement 306 can only conceal or cover a side of the sternal fixation wire 106 at the supplemented portion(s) 208, such that the supplemented portion(s) 208 include the covered side 214 and a bare side 216.

Additionally, the sternal fixation wire can include a first tying length 312, which can extend from the first dispersion supplement 302 in a direction opposite from the second dispersion supplement 306. The first tying length 312 of the sternal fixation wire 106 can omit force dispersion supplements 112 and can be configured for tying together with a second tying length 314 extending from the second dispersion supplement 306, in a direction opposite from the first dispersion supplement 302. The first tying length 312 can have a first length dimension 316 and/or the second tying length 314 can have a second length dimension 318 which provides additional wire for the surgeon to use to tie and tighten the sternal fixation wire 106. This wire tying procedure can be performed once the sternal fixation wire 106 has been placed around the first bone section 108 and the second bone section 110, and has been oriented with the first force dispersion supplement 302 and/or the second force dispersion supplement 306 pressed against the attachment zone(s) 118.

In some examples, force dispersion supplement(s) 112 can be statically fixed at their locations on the sternal fixation wire 106. Additionally or alternatively, the force dispersion supplement(s) 112 can be slidable and/or adjustable along the length of the sternal fixation wire 106. The sternal fixation device 102 can include a mechanism to lock the force dispersion supplement(s) 112 in place at a particular position on the sternal fixation wire 106 once the user has moved the force dispersion supplement(s) 112 to a desired location. This mechanism can include a crimping mechanism, application of an adhesive, or so forth.

As shown in FIGS. 3C and 3D, the bone fixation system 100 can include a plurality of force dispersion supplements 320 spaced apart by multiple bare portions 322. The multiple bare portions 322 can have a plurality of length dimensions 324. In some examples, the plurality of length dimensions 324 can be equal to each other, such that the plurality of force dispersion supplements 322 are evenly spaced apart. Additionally, the plurality of length dimensions 324 can have different values. For instance, a first length dimension 326 between the first force dispersion supplement 302 and the second force dispersion supplement 306 can be different than (e.g., greater than or less than) a second length dimension 328 between the second force dispersion supplement 306 and a third force dispersion supplement 330. Moreover, the second length dimension 328 between the second force dispersion supplement 306 and the third force dispersion supplement 330 can be different than (e.g., greater than or less than) a third length dimension 332 between the third force dispersion supplement 330 and a fourth force dispersion supplement 334. Also, the third length dimension 332 between the third force dispersion supplement 330 and the fourth force dispersion supplement 334 can be different than (e.g., greater than or less than) a fourth length dimension 336 between the fourth force dispersion supplement 334 and a fifth force dispersion supplement 338. These varying length dimensions of the multiple bare portions 322 can go on for any number of force dispersion supplements 320 (e.g., 8, 9, 10, 11 12, or so forth) disposed on the sternal fixation wire 106.

In this way, the differing length dimensions 324 between the plurality of force dispersion supplements 320 can be configured to align with different portions of the first bone section 108 and/or the second bone section 110 receiving the sternal fixation device 102. In other words, the lateral width dimension of the sternum 104 can vary between the different attachment zones 118, and the varying length dimensions of the multiple bare portions 322 can correspond to these variances in the sternum width. For instance, as depicted in FIG. 1, the lateral width dimension between the first attachment zones 130 can be greater than the lateral width dimension between the second attachment zones 146. Also, the lateral width dimension between the second attachment zones 146 can be less than the lateral width dimension between the third attachment zones 156. The lateral width dimension between the third attachment zones 156 can be less than the lateral width dimension between the fourth attachment zones 164. Moreover, the lateral width dimension between the fourth attachment zones 164 can be greater than the lateral width dimension between the fifth attachment zones 172.

Additionally, the plurality of force dispersion supplements 320 can have a plurality of length dimensions 340 which can be a same dimension such that the plurality of force dispersion supplements 320 have uniform length dimensions. Alternatively, the plurality of length dimensions 340 can vary with respect to each other to match the particular dimensions/contours of the particular attachment zones 118 to which the different force dispersion supplements 320 attach. Furthermore, as shown in FIG. 3D, the plurality of force dispersion supplements 320 can only cover a side of the sternal fixation wire 106 at the supplemented portion(s) 208, such that the supplemented portions 208 include the covered sides 214 and the bare sides 216.

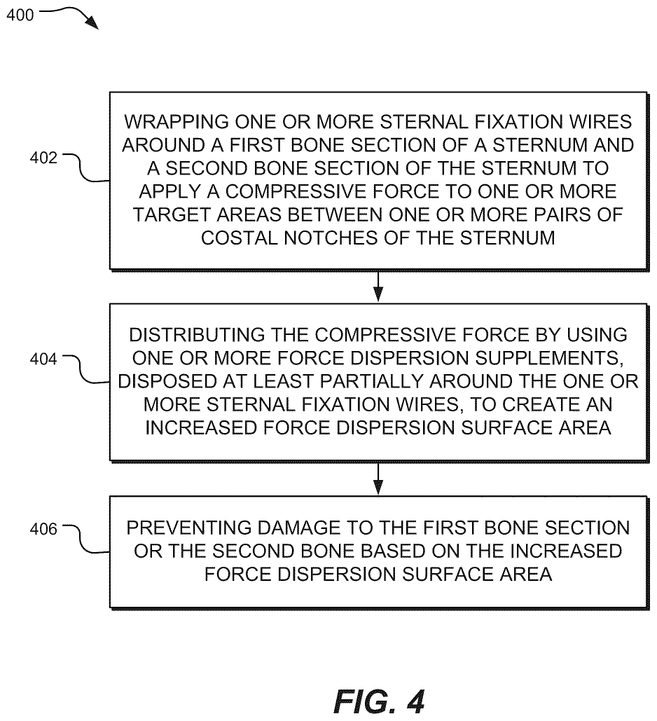

FIG. 4 depicts an example method 400 to heal two bone sections together, which can be performed with any of the system(s) 100 discussed herein regarding FIGS. 1-3D.

In some examples, at operation 402, the method 400 can wrap one or more sternal fixation wires around a first bone section of a sternum and a second bone section of the sternum to apply a compressive force to one or more target areas between one or more pairs of costal notches of the sternum. At operation 404, the method 400 can distribute the compressive force by using one or more force dispersion supplements, disposed at least partially around the one or more sternal fixation wires, to create an increased force dispersion surface area. At operation 406, the method 400 can prevent damage to the first bone section or the second bone based on the increased force dispersion surface area.

It is to be understood that the specific order or hierarchy of steps in the method(s) depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. Any components or features of one example can be combined with any components or features of any other example, These and other variations, modifications, additions, and improvements may fall within the scope of the disclosure as defined in the claims that follow.

What is claimed is:

1. A sternal fixation system comprising:
a sternal fixation wire operable to at least partially wrap around a first bone section and a second bone section to provide a compressive force on the first bone section and the second bone section, the compressive force pushing the first bone section and the second bone section toward a centerline between the first bone section and the second bone section; and
a force dispersion supplement disposed around at least a portion of the sternal fixation wire, the force dispersion supplement operable to increase a force dispersion surface area of the sternal fixation wire at an attachment zone on the first bone section or the second bone section.

2. The system of claim 1,
wherein,
the force dispersion supplement includes at least a flexible sleeve wrapped around the at least the portion of the sternal fixation wire.

3. The system of claim 1,
wherein,
the force dispersion supplement includes at a least a mesh pad.

4. The system of claim 3,
wherein,
the mesh pad includes interior space permitting blood to flow through the mesh pad.

5. The system of claim 1,
wherein,
the force dispersion surface area is increased at a sternal edge of a sternum body between a first costal notch and a second costal notch.

6. The system of claim 1,
wherein,
the force dispersion surface area is increased, by the force dispersion supplement, at a sternal edge of a manubrium between an articular facet for rib 1 and an articular demifacet for rib 2.

7. The system of claim 1,
wherein,
the force dispersion supplement covers a full circumferential surface of the at least the portion of the sternal fixation wire.

8. The system of claim 1,
wherein,
the force dispersion supplement conceals a first side of a circumferential surface of the at least the portion of the sternal fixation wire,
the first side mates with an outer surface of the first bone section or the second bone section, and
the forces dispersion supplement exposes a second side of the circumferential surface opposite the first side.

9. A sternal fixation system comprising:
one or more sternal fixation wires operable to at least partially wrap around a first bone section of a sternum and a second bone section of the sternum to provide a compressive force to the first bone section and the second bone section, the compressive force being applied to one or more attachment zones between one or more pairs of costal notches of the sternum; and
one or more force dispersion supplements disposed around one or more portion of the one or more sternal fixation wire, the one or more force dispersion supplements increase a force dispersion surface area applied to the one or more attachment zones.

10. The system of claim 9,
wherein,
the one or more sternal fixation wires includes:
a first sternal fixation wire having a first length corresponding to a first attachment zone of the one or more attachment zones, the first attachment zone being at least partially between a first costal notch and a second costal notch,
a second sternal fixation wire having a second length corresponding to a second attachment zone of the one or more attachment zones, the second attachment zone being at least partially between the second costal notch and a third costal notch, and
a third sternal fixation wire having a third length corresponding to a third attachment zone of the one or more attachment zones, the third attachment zone being at least partially between the third costal notch and a fourth costal notch.

11. The system of claim 10,
wherein,
the first length of the first sternal fixation wire is greater than the second length of the second sternal fixation wire, and
the one or more force dispersion supplements include:
a first force dispersion supplement disposed on the first sternal fixation wire, and
a second force dispersion supplement disposed on the second sternal fixation wire.

12. The system of claim 11,
wherein,
the third length of the third sternal fixation wire is greater than the second length of the second sternal fixation wire, and
the one or more force dispersion supplements include a third force dispersion supplement disposed on the third sternal fixation wire.

13. The system of claim 9,
wherein,
the one or more sternal fixation wires include a plurality of sternal fixation wires, and individual sternal fixation wires of the plurality of sternal fixation wires include:
a first force dispersion supplement of the one or more force dispersion supplements corresponding to a first sternal edge of the first bone section, and
a second force dispersion supplement of the one or more force dispersion supplements correspond to a second sternal edge of the second bone section.

14. The system of claim 9,
wherein,
the one or more sternal fixation wires includes one sternal fixation wire with two force dispersion supplements spaced a selectively determined distance apart along the one sternal fixation wire.

15. The system of claim 14,
wherein,
the one sternal fixation wire is a first sternal fixation wire,
the distance is a first distance, and
the sternal fixation system includes a second sternal fixation wire with two force dispersion supplements spaced a second distance apart along the second sternal fixation wire,
the second distance is different than the first distance.

16. A sternal fixation method comprising:
wrapping one or more sternal fixation wires around a first bone section of a sternum and a second bone section of the sternum to apply a compressive force to one or more target areas between one or more pairs of costal notches of the sternum;
distributing the compressive force by using one or more force dispersion supplements, disposed at least partially around the one or more sternal fixation wires, to create an increased force dispersion surface area; and
preventing damage to the first bone section or the second bone based on the increased force dispersion surface area.

17. The method of claim 16,
wherein,
the wrapping of the one or more sternal fixation wires includes orienting the one or more sternal fixation wires to mate the one or more force dispersion supplements with one or more attachment zones of the first bone section or the second bone section.

18. The method of claim 16, further comprising:
selectively concealing a first portion of the one or more sternal fixation wires with the one or more force dispersion supplements; and
selectively exposing a second portion of the one or more sternal fixation wires.

19. The method of claim 16, further comprising:
tying the one or more sternal fixation wires such that the first bone section is immobilized relative to the second bone section.

20. The method of claim 16:
wherein,
the wrapping of the one or more sternal fixation wires around the first bone section and the second bone section includes:
positioning a first force dispersion supplement of the one or more force dispersion supplements at a first attachment zone the first bone section including a first sternal edge, and
positioning a second force dispersion supplement of the one or more force dispersion supplements at a second attachment zone of the second bone section including a second sternal edge, across a center midline of the sternum from the first attachment zone.
